(19) United States
(12) Patent Application Publication
(10) Pub. No.: US 2005/0222415 A1
(43) Pub. Date: Oct. 6, 2005

(54) Process for the preparation of rosuvastatin

(76) Inventors: Yatendra Kumar, Gurgaon (IN); Shantanu De, New Delhi (IN); Mohammad Rafeeq, Pilibhit (IN); Hashim Nizar Poovanathil Nagoor Meeran, Pathanamthitta (IN); Swargam Sathyanarayana, Karim Nager (IN)

Correspondence Address: RANBAXY INC., 600 COLLEGE ROAD EAST, SUITE 2100, PRINCETON, NJ, 08540, US

(21) Appl. No.: 10/515,361

(22) PCT Filed: May 21, 2003
(86) PCT No.: PCT/IB03/01946

§ 371 Date: Apr. 25, 2005

(30) Foreign Application Priority Data
May 21, 2002 (IN) 575/DEL/2002

(51) Int. Cl. C07D 239/42

(52) U.S. Cl. 544/332

(57) ABSTRACT

The present invention relates to a cost effective and industrially advantageous process for the preparation of 4-4(fluorophenyl)-6-isopropyl-2-(N-methyl-N-methylsulphonylamino)-5-pyrimidinecarboxaldehyde, referred to here as pyrimidine aldehyde of structural Formula I and to the use of this compound as intermediate for the preparation of rosuvastatin.

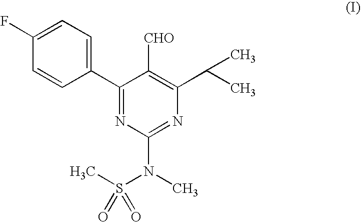

### FIELD OF THE INVENTION

The present invention relates to a cost effective and industrially advantageous process for the preparation of 4-4(fluorophenyl)-6-isopropyl-2-(N-methyl-N-methylsulphonylamino)-5-pyrimidinecarboxaldehyde, referred to here as pyrimidine aldehyde of structural Formula I 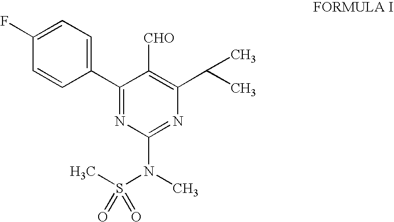
and to the use of this compound as intermediate for the preparation of rosuvastatin or a pharmaceutically acceptable salt thereof.

### BACKGROUND OF THE INVENTION

Chemically, rosuvastatin is (+)-(3R,5S)-7-[4-(4-fluorophenyl)-6-isopropyl-2-(N-methyl-N-methylsulphonylamino)pyrimidin-5-yl]-3,5-dihydroxy-6(E)-heptenoic acid calcium salt (2:1) having the structural Formula II 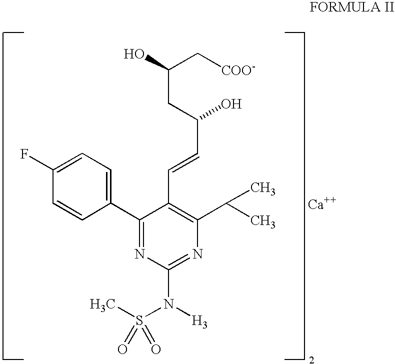
Rosuvastatin is an antihypercholesterolemic drug used in the treatment of atherosclerosis.

Hypercholesterolemia is now well recognized as a primary risk in coronary heat disease. Clinical studies with lipid lowering agents have established that decreasing elevated serum cholesterol level reduces the incidence of cardiovascular mortality. Recently, it has been found that rosuvastatin calcium has consistently shown greater potency than other currently marketed statins (atorvastatin, simvastatin and pravastatin) in preclinical and clinical testing.

Rosuvastatin and a process for its preparation are disclosed in U.S. Pat. No. 5,260,440. The process disclosed therein involves four distinct chemical steps: (1) condensation of methyl(3R)-3-(tert-butyldimethylsilyloxy)-5-oxo-6-triphenylphosphoranylidene hexanate, referred to here as phosphorane with 4-(4-fluorophenyl)-6-isopropyl-2-(N-methyl-N-methylsulfonylamino)-5-pyrimidinecarboxaldehyde, referred to here as pyrimidine aldehyde; (2) deprotection of the 3-hydroxyl group to give a keto alcohol; (3) reduction of 5-oxo to get a chiral dihydroxy heptenate; and (4) hydrolysis of the dihydroxy heptenate and conversion to hemicalcium salt.

The generation of the pyrimidine aldehyde requires eight synthetic steps and involves the use of expensive reagents and toxic solvents. The process results in the formation of several side products at various intermediate steps thus necessitating purification at the cost of low yields. The process is both uneconomical and time consuming, hence not suitable for commercial production.

It is, therefore, desirable to provide an efficient process for the preparation of rosuvastatin which improves the economics by employing less expensive reagents and is more productive.

### SUMMARY OF THE INVENTION

The present invention provides a process for the preparation of rosuvastatin, its salts, esters, or the corresponding cyclized lactone form. The process provides obvious benefits with respect to economics and convenience to operate on a commercial scale.

### DETAILED DESCRIPTION OF THE INVENTION

In accordance with one aspect, there is provided a process for the preparation of 4-(4-fluorophenyl)-6-isopropyl-2-(N-methyl-N-methylsulphonylamino)-5-pyrimidinecarboxaldehyde of structural Formula I as shown in Scheme I, 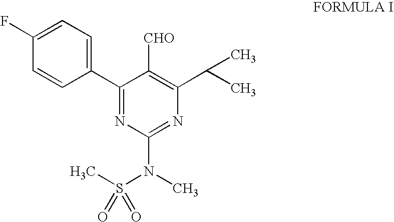 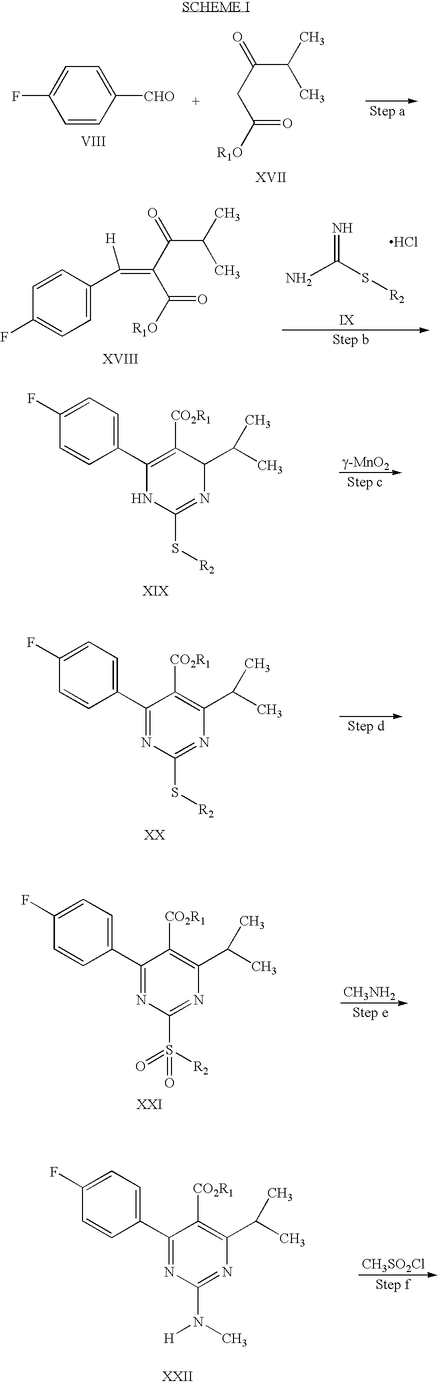 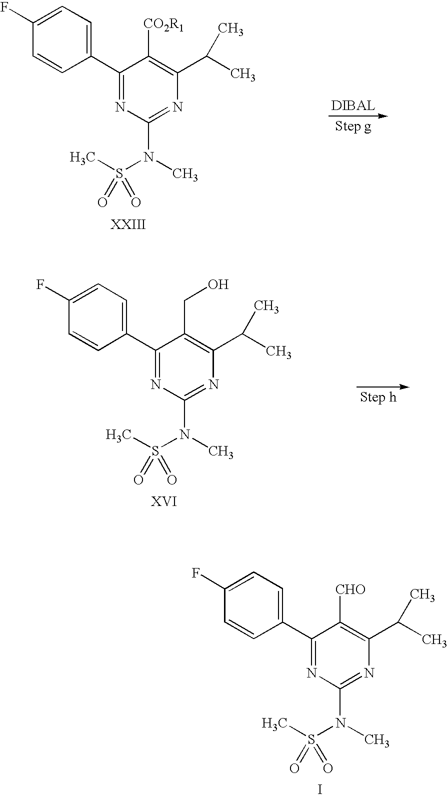
comprising:
a. condensing 4-fluorobenzaldehyde of structural Formula VIII with a compound of structural Formula XVII, wherein R₁ is independently C₂₋₆ alkyl, C₁₋₆ cycloalkyl or aralkyl, to give an olefin of structural Formula XVIII,
b. reacting the olefin with isothiourea of structural Formula IX, wherein R₂ is independently C₂₋₆ alkyl, C₁₋₆ cycloalkyl or aralkyl, to give a cyclized dihydropyrimidine derivative of structural Formula XIX,
c. aromatization of the dihydropyrimidine derivative with γ-manganese dioxide to give a pyrimidine compound of structural Formula XX,
d. oxidation of the pyrimidine compound to give a sulphonyl derivative of structural Formula XXI,
e. subjecting the sulphonyl derivative to methylamination to give an N-methylpyrimidine derivative of structural Formula XXII,
f. methanesulphonylation of the N-methylpyrimidine derivative to give an N-methyl methanesulphonamide derivative of structural Formula XXIII,
g. reduction of the N-methyl methanesulphonamide derivative with diisobutylaluminium hydride (DIBAL) in toluene to give an alcoholic compound of structural Formula XVI, and
h. oxidation of the alcoholic compound to give a pyrimidine aldehyde of structural Formula I.

The condensation at step a) can be carried out in a suitable solvent, for example hexane, heptane, cycloheptane, cyclohexane, and mixture(s) thereof at a reflux temperature in the presence of piperidine and glacial acetic acid.

The cyclization at step b) can be carried out in a suitable solvent, for example N, N-dimethylacetamide, N,N-dimethylformamide, dimethylsulphoxide, acetonitrile, and mixture(s) thereof in the presence of molecular sieves.

The aromatization at step c) can be carried out with β-manganese dioxide in the presence of a solvent, for example dichloromethane, chloroform toluene, benzene, ethyl acetate, and mixture(s) thereof.

The oxidation reaction at step d) can be carried out with peracetic acid or hydrogen peroxide in a solvent, for example dichloromethane, chloroform, toluene, benzene, ethyl acetate, and mixture(s) thereof.

The methylamination at step e) can be carried out with methylamine in a solvent, for example toluene, methylene chloride, tetrahydrofuran, dioxane, and mixture(s) thereof.

The methanesulphonation at step f) can be carried out in the presence of n-butyllithium.

The selective oxidation of the alcoholic compound at step h) can be carried out with γ-manganese dioxide in a suitable solvent, for example methylene chloride, tetrahydrofuran, dioxane, and mixture(s) thereof to give a pyrimidine aldehyde of structural Formula I.

The reactions (a) to (h) of Scheme I can be performed and worked up in a manner conventional for the type of reaction involved. The reaction parameters such as concentration, reaction duration, temperature, molar ratios of reagents can be chosen according to principles well established in the art.

In accordance with a second aspect, there is provided a process for the preparation of cyclized dihydropyrimidine derivative of structural Formula XIX comprising reaction of an olefin of structural Formula XVIII with isothiourea of structural Formula IX, wherein R₂ is independently C₂₋₆ alkyl, C₁₋₆ cycloalkyl or aralkyl.

In accordance with a third aspect, there is provided a process for the preparation of a pyrimidine compound of structural Formula XX comprising aromatization of the dihydropyrimidine derivative of structural Formula XIX with γ-manganese dioxide.

In accordance with a fourth aspect, there is provided a process for the preparation of a sulphonyl derivative of structural Formula XXI comprising oxidation of the pyrimidine compound of structural Formula XX with peracetic acid or hydrogen peroxide.

In accordance with a fifth aspect, there is provided a process for the preparation of an N-methylpyrimidine derivative of structural Formula XXII comprising reaction of the sulphonyl derivative of structural Formula XXI with methyl amine. The methylamination can be carried out in a solvent, for example toluene, methylene chloride, tetrahydrofuran, dioxane, and a mixture thereof.

In accordance with a sixth aspect, there is provided a process for the preparation of a pyrimidine aldehyde of structural Formula I comprising oxidation of alcoholic compound of structural Formula XVI with γ-manganese dioxide.

In another aspect, the pyrimidine aldehyde of Formula I prepared by the process of the present invention can be subjected to Wittig condensation with methyl(3R)-3-(tert-butyldimethylsilyloxy)-5-oxo-6-triphenylphosphoranylidene hexanate (phosphorane) of structural Formula III to provide a condensed product of structural Formula IV. The condensed product is deprotected with methanesulphonic acid to provide a keto alcohol of structural Formula V, which is further reduced to afford a dihydroxyheptenate of Formula VI, which is hydrolyzed to give rosuvastatin of structural Formula II as shown in Scheme II.

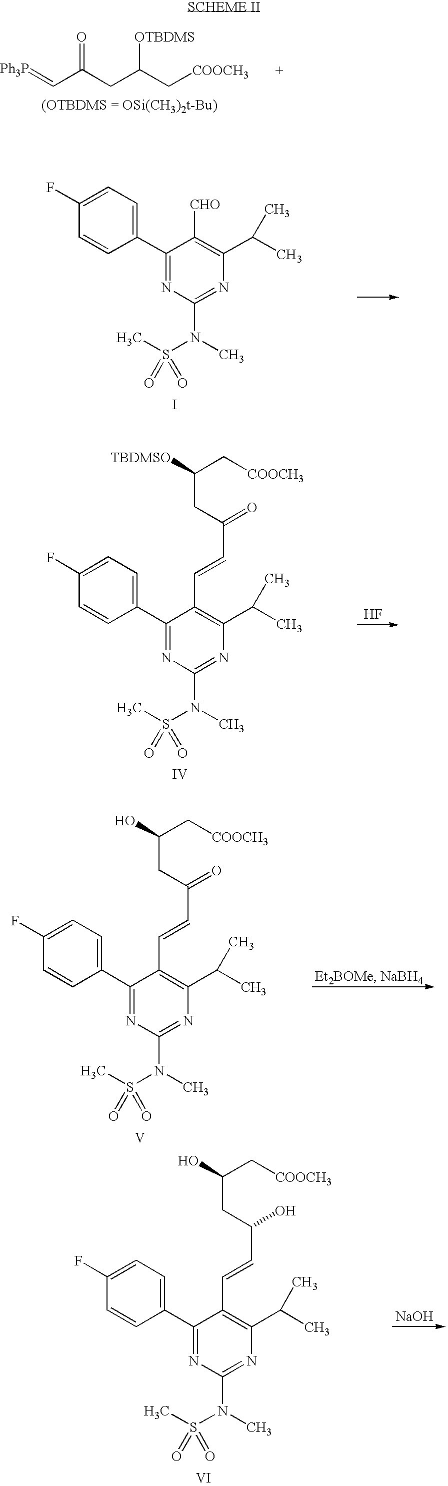

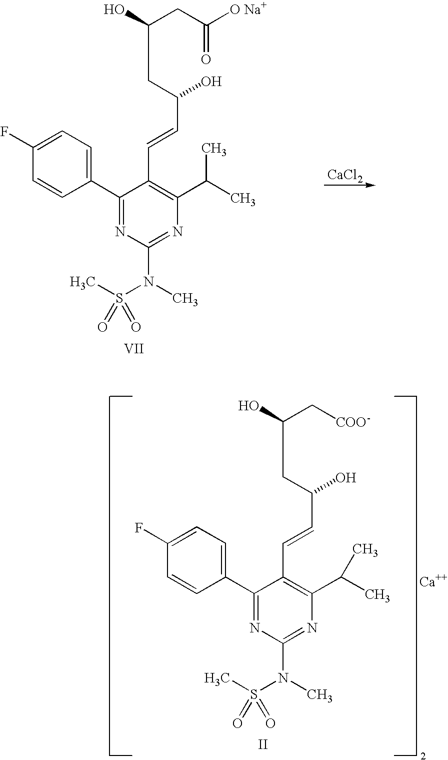

The starting material, methyl(3R)-3-(tert-butyldimethylsilyloxy)-5-oxo-6-triphenylphosphoranylidene hexanate of structural Formula III may be prepared by methods known in the literature, for example as described in U.S. Pat. No. 5,620,440.

Methods known in the art may be used with the process of this invention to enhance any aspect of the process. Any one familiar with organic process research development can do variations in various reaction parameters described above. The product obtained may be further purified by any technique known to a person skilled in the art, for example, by filtration, crystallization, column chromatography, preparative high pressure liquid chromatography, preparative thin layer chromatography, extractive washing in solution or a combination of these procedures.

The examples mentioned below demonstrate specific preparations of the present invention. The examples are provided to illustrate the details of the invention and should not be constrained to limit the scope of the present invention.

### EXAMPLE 1

Preparation Of 4-(4-Fluorophenyl)-6-isopropyl-2-(N-methylsulphonylamino)-5-pyrimidinecarboxaldehyde (I) (Pyrimidine Aldehyde)

### Step a—Preparation of Methyl 3-(4-fluorophenyl)-2-(2-methyl-1-oxopropyl)-prop-2-enoate (XVIII) (Olefin)

To a mixture of piperidine (1.06 gm, 0.18 mmoles equivalent) and glacial acetic acid (2.08 gm, 0.5 moles equivalent) in hexane (110 ml), was added 4-fluorobenzaldehyde (8.7 gm, 1.01 moles equivalent) and methylisobutyryl acetate (10 gm, 1 mole equivalent) at room temperature. The reaction mixture was heated to reflux with simultaneous removal of water azeotropically for 12-16 hours. After the reaction was over, the mixture was cooled and dimethylformamide (10 ml) was added. It was stirred and the organic portion was washed with 10% aqueous sodium metabisulphite, 5% dilute hydrochloric acid and 10% brine. The evaporation of the solvent gave olefin as a semi-solid.

Yield: 100% (GC Purity >98%).

### Step b—Preparation of Methyl 4-(4-fluoro-phenyl)-6-isopropyl-2-benzylthio-3h-pyrimidine-5-carboxylate (XIX) (Dihydropyrimidine Intermediate)

A mixture of the compound of Formula XVIII (75 g, 0.3 mole), S-benzylisothiourea hydrochloride (60.81 g, 0.3 mole) and molecular sieves (300 g) in N, N-dimethylformamide (375 ml) was stirred at room temperature for 610 hours and then slowly heated to 85° C.-90° C. for 6-10 hours. After the reaction was over, the mixture was cooled to room temperature and filtered through celite. The mixture was then washed with 5% sodium bicarbonate followed by 5% dilute hydrochloric acid and 10% brine. The organic portion was then concentrated and crystallized in isopropyl ether yielding the dihydropyrimidine intermediate (I) as a pure white solid.

Yield: 0.81% (HPLC quality >98%)

### Step c—Preparation of Methyl 4-(4-fluorophenyl)-6-isopropyl-2-benzylthio-pyrimidine-5-carboxylate (XX) (Pyrimidine Compound)

The dihydropyrimidine intermediate obtained above (108.0 g, 0.271 mole) and γ-MnO₂ (324 g) were taken in dichloromethane and the reaction mixture was stirred at 35° C. for 30 to 60 minutes. The reaction mixture was filtered through celite and the solvent removed to yield a solid product.

Yield: 100% (HPLC quality >97%)

### Step d—Preparation of Methyl 4-(4-fluorophenyl)-6-isopropyl)-6-isopropyl-2-benzylsulphonyl pyrimidine-5-carboxylate (XXI) (Benzenesulphonyl Intermediate)

To a solution of the pyrimidine compound, XX (45.0 g, 0.113 mole) in dichloromethane at 25° C.-28° C., was added peracetic acid (112.5 ml) drop wise. The reaction mixture was stirred for few hours at 33° C.-34° C. After the reaction was over, 5% aqueous solution of sodium hydrogen carbonate was added drop wise. The organic layer was separated and dried over anhydrous sodium sulphate. The title compound was isolated in methanol as a solid.

Yield: 95% (HPLC quality >99%).

### Step e—Preparation of Methyl 4-(4-fluorophenyl)-6-isopropyl-2-(n-methylamino)pyrimidine-5-carboxylate (XXI) (N-methyl Pyrimidine Intermediate)

The above obtained benzylsulphonyl intermediate (40.0 g, 0.0934 mole) was taken in dichloromethane (500 ml) and cooled to −10° C. to −15° C. A solution of methylamine (7.98 g, 0.249 mole) in dichloromethane was added drop wise under cooling. The mixture was stirred at ambient temperature for few hours. The solution was filtered and the filtrate was washed with water, organic layer was dried over anhydrous sodium sulphate and the product was isolated in hexane at 0° C.-5° C.

Yield: 100% (HPLC quality >99%)

### Step f—Preparation of Methyl 4-(4-fluorophenyl)-6-isopropyl-2-(n-methyl-n-methylsulphonylamino)pyrimidine-5-carboxylate (XI) (Sulphonamide Intermediate)

The intermediate N-methylpyrimidine (36.0 g, 0.11 mole) obtained above was taken in tetrahydrofuran (360 ml) and cooled to −70° C. to −75° C. A solution of N-butyllithium in hexane (1.6 molar, 150.0 ml) was added drop wise under cooling. The reaction mixture was stirred for a few hours followed by drop wise addition of methanesulphonyl chloride (30.6 g, 0.26 mole). After the reaction was over, water (340 ml) was added slowly and the tetrahydrofuran layer was concentrated to get an oily residue. This was taken in ethyl acetate (540 ml) and washed with 5% sodium bicarbonate and 10% brine. The solvent was evaporated to give crude N-methyl methanesulphonamide intermediate as a solid material. The isolation form diisopropyl ether gave the title compound as a pure solid product.

Yield: 63% (HPLC Quality >98%)

### Step g—Preparation of [4-(4-Fluorophenyl)-6-isopropyl-2-(N-methyl-N-methylsulphonylamino)pyrimidin-5-yl]methanol (XVI) (Pyrimidine Alcohol Intermediate)

To a solution of the above obtained compound (7.0 g, 0.0183 mole) in toluene (70 ml), was added drop wise DIBAL (20% solution in toluene; 47.6 ml) at −70° C. to −75° C. and stirred the solution or few hours. After the reaction was over, it was quenched with saturated ammonium chloride solution followed by filtration of the reaction mixture through celite. The filtrate was treated with charcoal and the solvent was removed to yield pyrimidine alcohol (XVI).

Yield: 80% (HPLC Quality >99%).

### Step h—Preparation of 4-(4-Fluorophenhl)-6-isopropyl-2-(N-methyl-N-methylsulphonylamino)-5-pyrimidinecarboxaldehyde (1) (Pyrimidine Aldehyde Intermediate)

To the above obtained pyrimidine alcohol (1 g), was added dichloromethane (10 ml) followed by the addition of γ-active manganese dioxide (6 g). The reaction mixture was stirred at room temperature for few hours followed by refluxing for 8-10 hours. After the reaction was over, it was filtered through celite. The removal of solvent followed by crystallization in toluene-ethyl acetate mixture gave pyrimidine aldehyde (I).

Yield: 80.5% (HPLC Quality >99%).

### EXAMPLE 2

Preparation of Rosuvastatin

### Step a—Preparation of Methyl 7-[4-(4-fluorophenyl)-6-isopropyl-2-(n-methyl-n-methylsulphonylamino)pyrimidin-5-yl]-(3r)-3-(tert-butyldimethyl silyloxy)-5-oxo-(e)-6 heptenate (V) (Protected Heptenate)

A solution of 100 g of pyrimidine aldehyde, 228 g of phosphorane, methyl(3R)-3-(tert-butyldimethylsilyloxy)-5-oxo-6-triphenylphosphoranylidene hexanate and 1500 ml of toluene was refluxed for about 30 hours and the reaction mixture was concentrated under reduced pressure. Cyclohexane (1500 ml) was added and the solution was cooled to 10° C. and stirred for 2 hours at 10° C.-12° C. The solution was filtered and concentrated under vacuum. The concentrate so obtained was dissolved in cyclohexane (1000 ml) and the residue was discarded. The solution so obtained was concentrated to 500 ml, cooled and filtered. The filtrate was concentrated under vacuum to give thick oil.

Yield: 100%

### Step b—Preparation of Methyl 7-[4-(4-fluorophenyl)-6-isopropyl-2-(n-methyl-n-methylsulphonylamino)-pyrimidin-5-yl]-(3r)-3-hydroxy-5-oxo-[e)-6-heptenate (V) (Keto Alcohol Intermediate)

To the compound of Formula TV (100 g) in methanol (1000 ml), was added a solution of methanesulphonic acid (10 g) in water (190 ml) at 15° C. The reaction mixture was stirred for 6 hours at 30° C.-35° C. and concentrated under vacuum. The residue was extracted with dichloromethane (750 ml), washed with water (300 ml) and with aqueous sodium bicarbonate (1% w/v; 300 ml). The solution was concentrated under vacuum to give thick oil.

Yield: 96%

### Step c—Preparation of (+)-(3r,5s), methyl 7-[4-(4-fluorophenyl)-6-isopropyl-2-(n-methyl-n-methylsulphonylamino)-pyrimidin-5-yl]-3,5-dihydroxy-6(e)-heptenate (VI) (Dihydroxy Heptenate)

To a solution of 13 g of the compound of Formula V in 350 ml of anhydrous tetrahydrofuran and 90 ml of methanol, was added a solution of 13 ml of 1M diethylmethoxyborane in tetrahydrofuran at −78° C., and the mixture was stirred at the same temperature for 30 minutes. To the mixture, was added 1.3 g of a sodium borohydride and the mixture was stirred for another 3 hours. Acetic acid (16 ml) was added thereto, and the mixture was adjusted to pH 8 with saturated sodium bicarbonate and extracted with ether. The organic layer was washed with water, dried and evaporated ether under reduced pressure. To the resulting residue, methanol was added and the mixture was evaporated under reduced pressure. The resulting residue was subjected to column chromatography of silica gel eluting with methylene chloride/ether (3:1) to give 11.4 g of dihydroxy compound, methyl 7-[4-fluorophenyl)-6-isopropyl-2N-methyl-N-methylsulphonylamino)pyrimidin-5-yl]-(3R,5S)-dihydroxy-(E)-6-heptenate (VI) as syrup.

Yield: 85.2%

### Step d—Preparation of (+)-(3r,5s)-7-[4-(4-fluorophenyl)-6-isopropyl-2-(N-methyl-N-methylsulphonylamino)-pyrimidin-5-yl]-3,5-dihydroxy-6(e)-heptenoic acid sodium salt (VII) (Rosuvastatin Sodium Salt)

To a solution of 11.4 g of the compound of Formula VI in 160 ml of ethanol, was added 100 ml of 0.25 N sodium hydroxide under ice-cooling. The reaction mixture was warmed to room temperature and stirred for 1 hour. The solution was concentrated to remove the organic solvent under vacuum at 35° C.-40° C. The aqueous layer was washed with methyl tertiary butyl ether (MTBE) (500×2 ml) twice and filtered through a hyflo bed. The aqueous layer was used as such in the next step.

### Step e—Preparation of (+)-(3r,5s)-7-[4-(4-fluorophenyl)-6-isopropyl-2-(N-methyl-N-methylsulphonylamino)-pyrimidin-5-yl]-3,5-dihydroxy-6(e)-heptenoic acid calcium salt (D) (Rosuvastatin Calcium Salt)

To the aqueous solution of rosuvastatin sodium obtained from the previous step, was added an aqueous solution of calcium acetate and the reaction mixture was stirred for 2 hours at room temperature. The reaction mixture was filtered, washed with water (100 ml) and dried at 40° C.-45° C. under vacuum for 6 hours to get 9.0 g of rosuvastatin calcium.

Yield: 80.7%

While the present invention has been described in terms of its specific embodiments, certain modifications and equivalents will be apparent to those skilled in the art and are intended to be included within the scope of the present invention.

1. A process for the preparation of 4-(4-fluorophenyl)-6-isopropyl-2-(N-methyl-N-methylsulphonylamino)pyrimidin-5-yl]carboxaldehyde of structural Formula I, 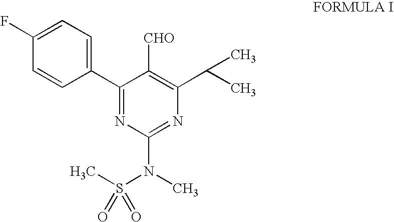
comprising:
a. condensing 4-fluorobenzaldehyde of structural Formula VIII 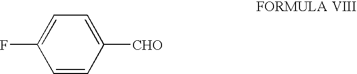
with a compound of structural Formula XVII, wherein R₁ is independently C₂₋₆ alkyl, C₁₋₆ cycloalkyl or aralkyl, 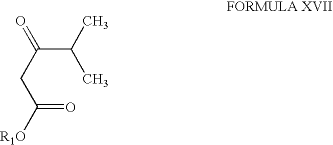
to give an olefin of structural Formula XVIII, 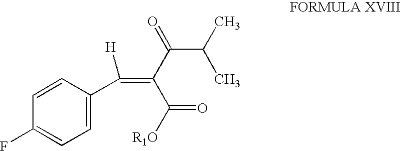
b. reacting the olefin with isothiourea of structural Formula IX, wherein R₂ is independently C₂₋₆ alkyl, C₁₋₆ cycloalkyl or aralkyl, 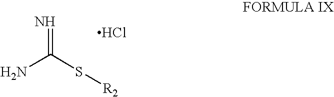
to give a cyclized dihydropyrimidine derivative of structural Formula XIX, 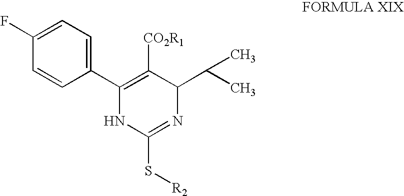
c. aromatizing the dihydropyrimidine derivative with γ-manganese dioxide to give a pyrimidine compound of structural Formula XX, 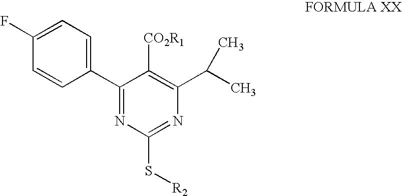
d. oxidizing the pyrimidine compound to give a sulphonyl derivative of structural Formula XXI, 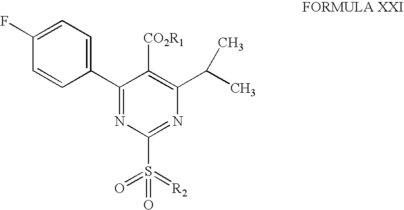
e. reacting the sulphonyl derivative with methylamine to give an N-methylpyrimidine derivative of structural Formula XXII, 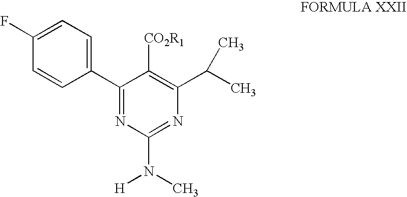
f. reacting the N-methylpyrimidine derivative with methanesulphonyl chloride to give an N-methyl methanesulphonamide derivative of structural Formula XXIII, 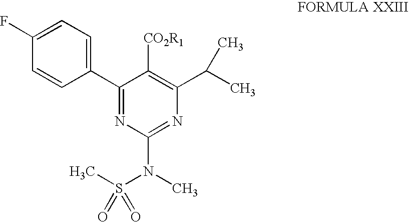
g. reducing the N-methyl methanesulphonamide derivative with diisobutylaluminum hydride (DIBAL) in toluene to give an alcoholic compound of structural Formula XVI, and 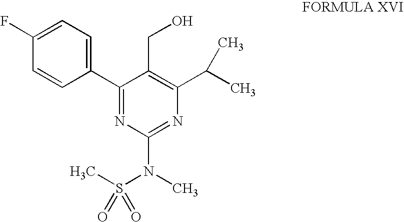
h. oxidizing the alcoholic compound to give a pyrimidine aldehyde of structural Formula I.

2. The process according to claim 1, wherein step (a) is carried out in a suitable solvent at reflux temperature in the presence of piperidine and glacial acetic acid.

3. The process according to claim 2, wherein the solvent is selected from the group consisting of hexane, heptane, cyclopentane, cyclohexane, and mixture(s) thereof.

4. The process according to claim 3, wherein the solvent is hexane.

5. The process according to claim 1, wherein step (b) is carried out in the presence of molecular sieves.

6. The process according to claim 1, wherein step (b) is carried out in a suitable solvent.

7. The process according to claim 6, wherein the solvent is selected from the group consisting of N,N-dimethylacetamide, N,N-dimethylformamide, dimethylsulphoxide, acetonitrile, and mixture(s) thereof.

8. The process according to claim 1, wherein step (c) is carried out in a solvent.

9. The process according to claim 8, wherein the solvent is selected from the group consisting of dichloromethane, chloroform, toluene, benzene, ethyl acetate, and mixture(s) thereof.

10. The process according to claim 1, wherein step (d) is performed with peracetic acid or hydrogen peroxide.

11. The process according to claim 11, wherein step (d) is performed with peracetic acid.

12. The process according to claim 1, wherein step (d) is carried out in a solvent.

13. The process according to claim 12, wherein the solvent is selected from the group consisting of dichloromethane, chloroform, toluene, benzene, ethyl acetate, and mixture(s) thereof.

14. The process according to claim 1, wherein step (e) is carried out in a solvent.

15. The process according to claim 14, wherein the solvent is selected from the group consisting of toluene, methylene chloride, tetrahydrofuran, dioxane, and mixture(s) thereof.

16. The process according to claim 1, wherein step (f) is performed in the presence of n-butyl lithium.

17. The process according to claim 1, wherein step (h) is carried out in the presence of γ-manganese dioxide.

18. The process according to claim 1, wherein step (h) is carried out in a solvent.

19. The process according to claim 17, wherein the solvent is selected from the group consisting of methylene chloride, tetrahydrofuran, dioxane, and mixture(s) thereof.

20. A process for the preparation of cyclized dihydropyrimidine derivative of structural Formula XIX, wherein R₁ and R₂ are independently C₂₋₆ alkyl, C₁₋₆ cycloalkyl or aralkyl, 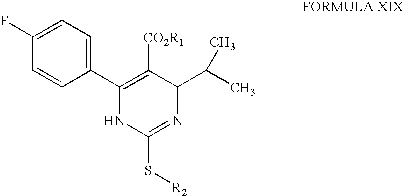
comprising reacting an olefin of structural Formula XVIII, wherein R₁ is as defined earlier, 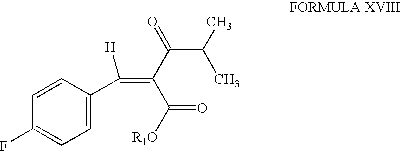
with isothiourea of structural Formula IX, wherein R₂ is as defined earlier.

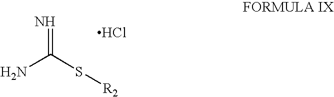

21. The process of claim 20, wherein the isothiourea is S-benzylisothiourea.

22. A process for the preparation of a pyrimidine compound of structure Formula XX, wherein R₁ and R₂ are independently C₂₋₆ alkyl, C₁₋₆ cycloalkyl or aralkyl, 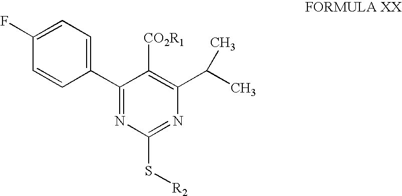
comprising aromatizing a dihydropyrimidine derivative of structural Formula XIX, wherein R₁ and R₂ are as defined earlier, with γ-manganese dioxide.

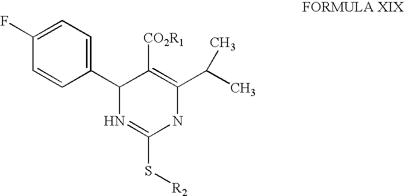

23. A process for the preparation of a sulphonyl derivative of structural Formula XXI, wherein R₁ and R₂ are independently C₂₋₆ alkyl, C₁₋₆ cycloalkyl or aralkyl, 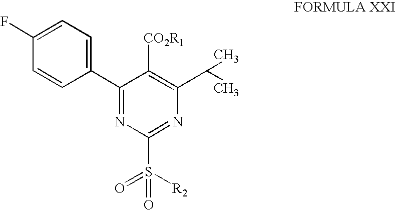
comprising oxidizing a pyrimidine compound of structural Formula XX, wherein R₁ and R₂ are as defined earlier, with peracetic acid or hydrogen peroxide.

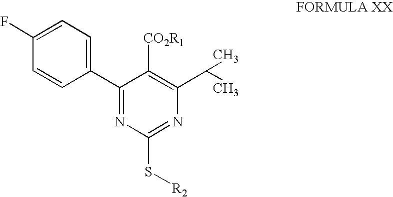

24. The process of claim 23, wherein the pyrimidine compound of structural Formula XX is oxidized with peracetic acid.

25. A process for the preparation of an N-methylpyrimidine derivative of structural Formula XXII, wherein R₁ and R₂ are independently C₂₋₆ alky, C₁₋₆ cycloalkyl or aralkyl, 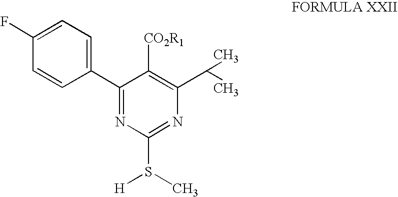
comprising reacting a sulphonyl derivative of structural Formula XXI, wherein R₁ is as defined earlier and R₂ is independently C₂₋₆ alkyl, C₁₋₆ cycloalkyl or aralkyl, with methylamine.

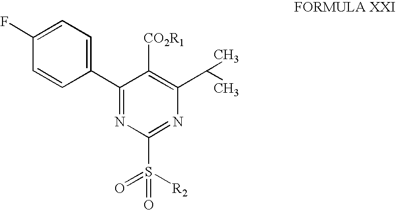

26. The process according to claim 25, wherein the reaction is carried out in a solvent.

27. The process according to claim 26, wherein the solvent is selected from the group consisting of toluene, methylene chloride, tetrahydrofuran, dioxane, and mixture(s) thereof.

28. A process for the preparation of 4-(4-fluorophenyl)-6-isopropyl-2-(N-methyl-N-methylsulphonylamino)pyrimidin-5-yl]carboxaldehyde of structural Formula I, 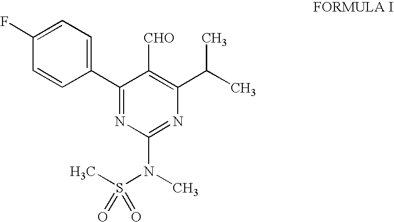
comprising oxidizing alcoholic compound of structural Formula XVI, 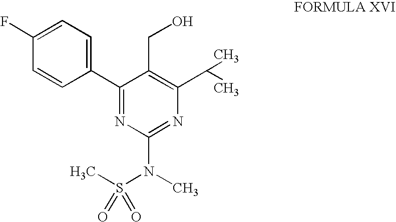
with γ-manganese dioxide to give the compound of structural Formula I.

29. A process for the preparation of rosuvastatin or a pharmaceutically acceptable salt thereof of structural Formula II, 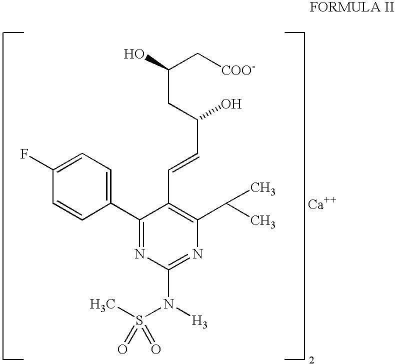
comprising:
a. condensing 4-fluorobenzaldehyde of structural Formula VIII 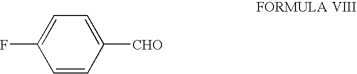
with a compound of structural Formula XVII, wherein R₁ is independently C₂₋₆ alkyl, C₁₋₆ cycloalkyl or aralkyl, 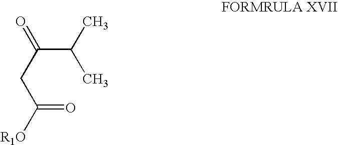
to give an olefin of structural Formula XVIII, 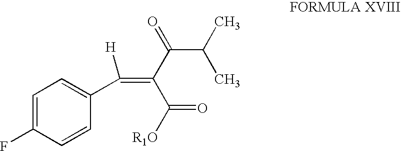
b. reacting the olefin with isothiourea of structural Formula IX, wherein R₂ is independently C₂₋₄ alkyl, C₁₋₆ cycloalky or aralkyl, 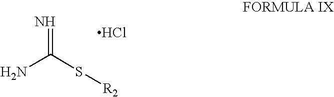
to give a cyclized dihydropyrimidine derivative of structural Formula XIX, 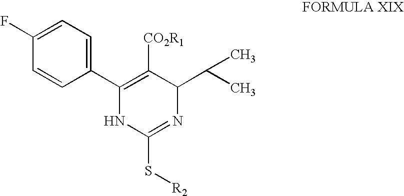
c. aromatizing the dihydropyrimidine derivative with γ-manganese dioxide to give a pyrimidine compound of structural Formula XX, 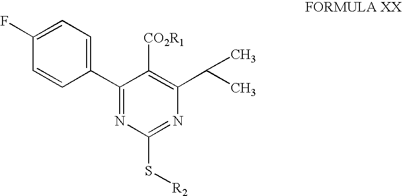
d. oxidizing the pyrimidine compound to give a sulphonyl derivative of structural Formula XXI, 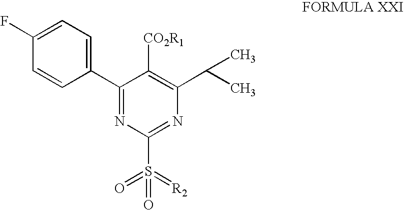
e. reacting the sulphonyl derivative with methylamine to give an N-methylpyrimidine derivative of structural Formula XXII, 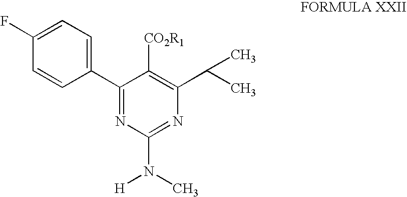
f. reacting the N-methylpyrimidine derivative with methanesulphonyl chloride to give an N-methyl methanesulphonamide derivative of structural Formula XXIII, 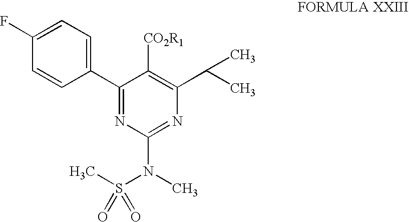
g. reducing the N-methyl methanesulphonamide derivative with diisobutylaluminium hydride (DIBAL) in toluene to give an alcoholic compound of structural Formula XVI, 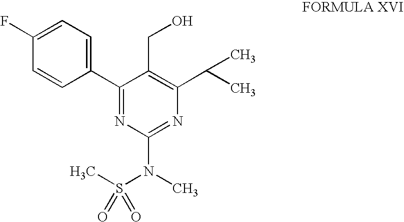
h. oxidizing the alcoholic compound to give a pyrimidine aldehyde of structural Formula I, 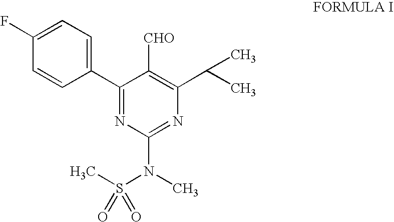
i. condensing the compound of structural Formula I with methyl(3R)-3-(tert-butyldimethylsilyloxy)-5-oxo-6-triphenyl-phosphoranylidene hexanate of structural Formula III, 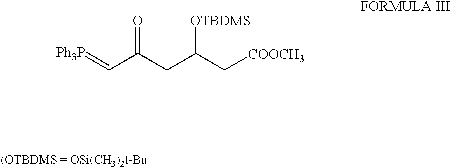
to give a condensed product of structural Formula IV, 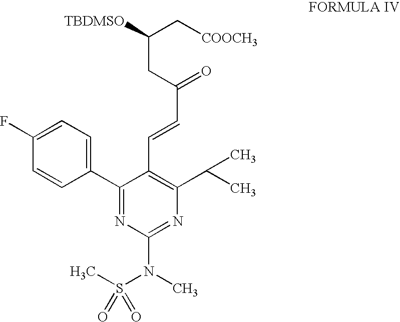
j. deprotecting the condensed product with methanesulphonic acid to give a keto alcohol of structural Formula V, 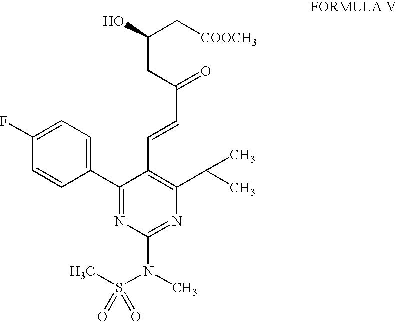
k. reducing the keto alcohol to give a dihydroxyheptenate of structural Formula VI, and 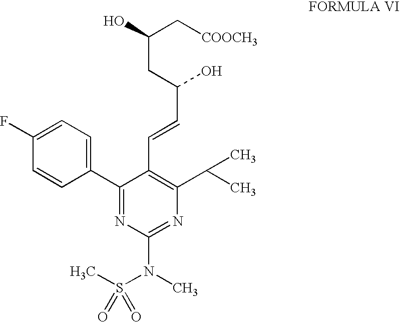
l. hydrolyzing the dihydroxyheptenate to give rosuvastatin of structural Formula II.

30. The process according to claim 29, wherein step (h) is carried out in the presence of γ-manganese dioxide.
